(19) United States
(12) Patent Application Publication
(10) Pub. No.: US 2013/0149701 A1
(43) Pub. Date: Jun. 13, 2013

(54) METHODS OF IDENTIFYING THERAPEUTIC AGENTS FOR TREATING PERSISTER AND BACTERIAL INFECTION

(71) Applicants: Ying Zhang, Baltimore, MD (US); Wanliang Shi, Baltimore, MD (US)

(72) Inventors: Ying Zhang, Baltimore, MD (US); Wanliang Shi, Baltimore, MD (US)

(21) Appl. No.: 13/810,663

(22) PCT Filed: Jun. 17, 2011
(86) PCT No.: PCT/US11/40926

§ 371 Date: Jan. 16, 2013

Related U.S. Application Data
(60) Provisional application No. 61/355,786, filed on Jun. 17, 2010.
(60) Provisional application No. 61/490,295, filed on May 26, 2011.

(51) Int. Cl. C12Q 1/18; G01N 33/573; G01N 33/566

(52) U.S. Cl. CPC C12Q 1/18; G01N 33/566; G01N 33/573
USPC 435/6.1; 436/501; 435/7.4; 435/7.1; 435/32

(57) ABSTRACT

The present invention relates to methods, compositions, assays and kits for identifying an antibacterial agent that decreases persister formation or survival, eliminates or reduces bacterial infection or disease and/or increases killing of a bacterial cell.

The research resulting in the invention described herein was supported in part by funding from the National Institutes of Health AI44063. The United States Government has certain rights in the invention.

### BACKGROUND

Pyrazinamide (PZA) is an important first-line tuberculosis (TB) drug that is most commonly used in combination with isoniazid and rifampin for the treatment of tuberculosis. PZA plays a unique role in shortening the tuberculosis treatment from previously 9-12 months to 6 months as a result of its ability to kill a population of persister M. tuberculosis bacteria that are not killed by other TB drugs.

Persisters pose a significant challenge to the control of various bacterial infections, as they underlie latent infections, chronic and recurrent infections, biofilm infections, lengthy therapy of certain bacterial infections as in tuberculosis and post-treatment persistence and relapse (e.g., Zhang, Y., Persistent and dormant tubercle bacilli and latent tuberculosis. Front Biosci, 2004. 9: p. 1136-56; McDermott, W., Microbial persistence. The Yale J Biol Med, 1958. 30: p. 257-91; and Lewis, K., Persister cells, dormancy and infectious disease. Nat Rev Microbiol, 2007. 5(1): p. 48-56). Persister bacteria pose enormous public health problems. The persister tubercle bacilli (TB) present a tremendous challenge for effective TB control and underlie the lengthy TB therapy. This makes patient compliance very difficult and is in part responsible for the increasing emergence of drug resistant TB such as the recently reported extreme drug resistant TB (XDR-TB) (J. Cohen, Science 313, 1554 (2006). Identifying how drugs like PZA that kill persister bacteria is key to finding new generation of persister antibiotics.

Moreover, most strains of M. tuberculosis that are resistant to PZA are due to mutations in the gene pncA encoding pyrazinamidase/nicotinamidase that is involved in conversion of PZA to pyrazinoic acid (POA), but a few PZA-resistant M. tuberculosis strains do not have pncA mutations, suggesting new mechanism of resistance.

Despite the importance of PZA in shortening the treatment of TB, its mechanism of action is the least understood of all tuberculosis drugs, and the target of PZA or its active metabolite, pyrazinoic acid (POA), remains elusive. Previous attempts to identify the mode of PZA action by genetic approaches have so far been unsuccessful. This invention identified a number of drug targets of PZA which can be used for identifying new antibiotics for treatment of TB infection and other bacterial infections and for identifying PZA-resistant TB bacteria.

There is a need for more effective sterilizing drugs, methods and compositions for treating persistent bacterial infections such as tuberculosis.

### BRIEF DESCRIPTION OF THE DRAWINGS

FIG. 1. Mycobacterial lysates were loaded onto the POA-linked and control columns and the proteins that bound to POA (A) and the control column (B) were analyzed by SDS-PAGE. Lane M, protein ladder; Lane 1, whole cell lysate; 2, flow-through fraction; 3, washing fraction; 4, elution fraction. The band indicated by the red arrow is RpsA.

FIG. 2. Concentration-dependent inhibition of trans-translation by POA with M. tuberculosis ribosome and DHFR template with rare codon cluster. This bar graph represents densitometry scan of band intensities of similar experiments in FIG. 4C performed 5 times (P value <0.024, n=5).

FIG. 3. Structures of POA derivative (5-hydroxyl-2-pyrazinecarboxylic acid) (A) and the control compound ethanolamine (B) coupled to Sepharose 6B column for the identification of POA binding proteins from M. tuberculosis.

FIG. 4. RpsA alignment and isothermal titration calorimetry (ITC) titration of RpsA and POA. (A) Alignment of RpsA from M. tuberculosis H37Rv, M. tuberculosis PZA-resistant strain DHM444 and M. smegmatis. R1 to R4 represent the four homologous RNA-binding domains in RpsA. Colored vertical lines in gray boxes indicate sequence variations in the highly conserved RpsA sequences compared with the wild type M. tuberculosis sequence. The expanded region shows the variability in amino acid sequence in the C-terminus of RpsA among mycobacterial species. The red arrow at position 438 amino acid residue indicates the deletion of alanine in the C-terminal region of the mutant RpsA. ITC binding studies indicate POA bound to the M. tuberculosis H37Rv RpsA (WT) (B, inset VI), but not DHM444 RpsA (Mutant) (Inset, IV), and only weakly with the M. smegmatis RpsA (M. smeg) (Inset II). PZA did not bind to wild type RpsA (Inset V) or mutant RpsA (Inset III). The lower panel of the FIG. 2B shows the typical molar ratio saturation plot of POA with wild type Mtb RpsA.

FIG. 5. (A) Concentration-dependent inhibition of tmRNA binding to wild type M. tuberculosis RpsA by POA (Lanes 2-7). tmRNA from M. tuberculosis was used as RNA alone control (Lane 1). The wild type RpsA interaction with tmRNA was not affected by PZA (200 g/ml) (Lane 8) or INH (1 g/ml) (Lane 9). (B) tmRNA had impaired binding to the mutant RpsA (Lane 2), and POA at different concentrations did not inhibit the interaction of the DHM444 mutant RpsA with tmRNA (Lanes 3-7); The mutant RpsA interaction with tmRNA was not affected by PZA (200 g/ml) (Lane 8) or INH (1 g/ml) (Lane 9). (C)POA at 100, 50, and 25 μg/ml inhibited trans-translation of the DHFR product in a concentration-dependent manner in the in vitro system that contained ribosomes from M. tuberculosis, tmRNA and recombinant SmpB from M. tuberculosis, template pDHFR-8×AGG rare codons that are required for trans-translation (Lanes 1-5). Arrowheads indicate the trans-translation product DHFR was still present with low concentration of POA at 12.5 μg/ml (Lane 4) or in the absence of POA (Lane 5). POA at different concentrations did not inhibit canonical translation in in vitro translation system using ribosomes from M. tuberculosis, template pDHFR with stop codon (D, Lanes 6-10), nor the trans-translation of DHFR using ribosome from M. smegmatis (E, Lanes 1-4), or using ribosome from E. coli (F, Lanes 1-4) in the trans-translation system that contained tmRNA and recombinant SmpB from M. tuberculosis, template pDHFR-8×AGG rare codons.

FIG. 6. A new model for the mode of action of PZA. PZA is converted to the active form POA by M. tuberculosis PZase intracellularly and inhibits targets including RpsA. Upon stress, translating ribosomes are stalled and incomplete polypeptides may be toxic to the cell. The bacterial cell resolves this problem by adding tmRNA to the stalled mRNA. tmRNA binds to SmpB and EF-Tu, activating the complex for ribosome interaction. The alanyl-tmRNA/SmpB/EF-Tu complex recognizes stalled ribosomes at the 3′ end of an mRNA without stop codon or with rare codons. Translation resumes using tmRNA as a message, resulting in addition of the tmRNA-encoded peptide tag to the C-terminus of the stalled polypeptide. The tagged protein and mRNA are then degraded by proteases and RNases, leading to rescue of stalled ribosomes. POA binding to RpsA interferes with the interaction of RpsA with tmRNA required for trans-translation. POA blockade of the trans-translation pathway leads to a defect in stalled ribosome rescue and depletion of available ribosomes and perhaps increased accumulation of toxic or deleterious proteins, ultimately affecting persister survival under stress conditions.

FIG. 7. POA binding studies.

FIG. 8. Purification of M. tuberculosis Endonuclease IV. Lane M: molecular weight marker; Lane 1, 2, 3: varying amounts of purified Endo IV.

FIG. 9. Endonuclease IV activity on pUC19 plasmid DNA as a substrate. Lane 1: a control without enzyme; Lane 2: Endo IV (Rv0670); Lane 3: Endo IV from E. coli; Lane 4: Endo V from E. coli.

FIG. 10. Endonuclease IV (Rv0670) activity is inhibited by POA but not by PZA or INH with pUC19 plasmid DNA as substrate. Lane1, control without enzyme Endo IV (Rv0670); Lane 2, sample with enzyme Endo IV (Rv0670) but without compound; Lane 3, sample with Endo IV (Rv0670) in the presence of 25 mM POA; Lane 4, sample with Endo IV (Rv0670) in presence of 25 mM PZA; Lane 5, sample with Endo IV (Rv0670) in presence of 25 mM INH; Lane 6, DMSO control.

FIG. 11. Endonuclease IV (Rv0670) exhibited endonuclease activity on AP site (A) and deaminated site U (B) or H(C) and their specific inhibition by POA but not INH. After the reaction with Rv0670 (1 pmol) at 37° C. for 5 hr, two small fragments were released on the 7 M urea denaturing 20% polyacrylamide gel. Lane 1: an oligo marker (31 bp, 20 bp, and 16 bp). Lane 2: positive control without POA. Lanes 3, 4, 5, 6, 7 and 8: POA added to the reaction at 2, 4, 8, 16, 25 and 34 mM concentrations. Lane 9 and Lane 10: samples incubated with INH and PZA at 25 mM.

### DESCRIPTION

Described herein are methods, compositions and assays for identifying or screening for an antibacterial agent that decreases persister infection or formation, eliminates or reduces bacterial or pathogen infection or disease and/or increases killing of a bacterial cell. In particular embodiments, the method includes the steps of: (a) contacting a test agent with a composition comprising a Pyrazinamide (PZA)-sensitive target protein; and b) determining whether the test agent binds to, or inhibits activity of, the target protein. In certain embodiments, binding of the target protein or inhibition of the target protein activity is indicative of a potential antibacterial agent for decreasing persister formation or infection, eliminating or reducing bacterial infection including latent infection or disease and/or increasing killing of a bacterial cell including persister bacterial cell.

In some embodiments, the test agent may be an agonist or an antagonist. An “agent” is understood herein to include a therapeutically active compound or a potentially therapeutic active compound. An agent can be a previously known or unknown compound. An agent can be selected or synthesized based on the known structure of PZA or an analogue thereof, or may be part of a combinatorial library or a compound library of known and/or unknown chemical compounds. Agents can also be selected based on their ability to mimic PZA activity or expression.

An “agonist” is understood herein as a chemical substance capable of initiating the same reaction or activity typically produced by the binding of an endogenous substance to its receptor. An “antagonist” is understood herein as a chemical substance capable of inhibiting the reaction or activity typically produced by the binding of an endogenous substance (e.g., an endogenous agonist) to its receptor to prevent signaling through a receptor or to prevent downstream signaling that is the normal result of activation of the receptor. The antagonist can bind directly to the receptor or can act through other proteins or factors required for signaling.

As used herein, a test agent may be a compound or molecule that mimics the in vivo or in vitro activity of Pyrazinamide (PZA), an active component of PZA, Pyrazinoic Acid (POA), or an analog thereof, for example, 5-hydroxyl-2-pyrazinecarboxylic acid or 6-hydroxyl-2-pyrazinecarboxylic acid. The activity of PZA can be its ability to bind and inhibit certain proteins from a bacterial cell or inhibit the metabolic process of a bacterial cell. As used herein, a target protein may be a protein derived from a bacterial cell that Pyrazinamide (PZA), an active component of PZA, Pyrazinoic Acid (POA), or an analog thereof, for example, 5-hydroxyl-2-pyrazinecarboxylic acid, binds to, or a protein that inhibits the metabolic activity of in vivo or in vitro. The target protein may be referred to as Pyrazinamide (PZA)-sensitive target protein, an active component of PZA-sensitive target protein, Pyrazinoic Acid (POA)-sensitive target protein, or 5-hydroxyl-2-pyrazinecarboxylic acid or 6-hydroxyl-2-pyrazinecarboxylic acid-sensitive target protein.

Examples of target proteins include, but are not limited to, Endonuclease IV (Rv0670) (NP_{—}215184) Nfo (end), Polynucleotide phosphorylase (Rv2783c) (NP_{—}217299) GpsI (PNPase) GpsI (PNPase) (a bifunctional enzyme with a phosphorolytic 3′ to 5′ exoribonuclease activity and a 3′-terminal oligonucleotide polymerase activity involved in mRNA processing and degradation in bacteria), Iron-regulated heparin-binding hemagglutinin (Rv0475) (NP_{—}214989) HbhA, 30S ribosomal protein S1 (Rv1630) (NP_{—}216146) RpsA, 30S ribosomal protein S4 (Rv3458c) (NP_{—}217975) RpsD, 50S ribosomal protein L9 (Rv0056) (NP_{—}214570) RplI, 50S ribosomal protein L10 (Rv0651) (NP_{—}215165) RplJ, 50S ribosomal protein L7/L12 (Rv0652) (NP_{—}215166) RplL, 50S ribosomal protein L29 (Rv0709) (NP_{—}215223) RpmC, Putative DNA repair ATPase (Rv2731) (NP_{—}217247), or Hypothetical protein (Rv3169) (NP_{—}217685).

In a particular embodiment, the target protein is derived from RNA degradosome, and the activity is degradation of mRNA. In some aspects, the target protein comprises components selected from one or more of RNase E, PNPase, RhlB, and enolase. As used herein, binding may occur in a variety of manners, e.g., covalent or ionic, and may be referred to as attaching, cross-linking or affecting the configuration or ability of the protein to perform its chemical or biological function in vivo or in vitro. Binding of the target protein may occur at a variety of points. In particular embodiments, the target protein is 30S ribosomal protein S1 (Rv1630) RpsA, and binding occurs on the C-terminus, for example at alanine residue 438 (ΔA438) in the C-terminus.

Activity, as used herein, refers to metabolic activity of a bacterial cell. Examples of activity include, but are not limited to, Endonuclease IV and V activity, DNA repair, RNA degradation, starvation survival, iron-regulated heparin-binding, e.g., involved in dissemination of M. tuberculosis in vivo, polynucleotide phosphorylization and ppGpp production, and translation process, trans-translation process, or both.

In some embodiments, the binding or activity may be direct or indirect on the target protein or the metabolic process. In some embodiments, the agent inhibits the function of a target protein indirectly. In certain aspects, the test agent inhibits EF-Tu function, SmpB function, and/or tmRNA (SsrA) function, all of which are involved in trans-translation process. In some aspects, the agent binds to 30S ribosomal protein S1 (Rv1630) RpsA and inhibits EF-Tu, tmRNA (SsrA) or SmpB function involved in trans-translation process.

In yet further embodiments, a method is provided for identifying PZA resistance in a bacterial cell, comprising the steps of: (a) determining PZA-sensitivity, e.g., binding or inhibitory activity, in a wild type strain of said bacterial cell, and (b) determining PZA-sensitivity, e.g., binding or inhibitory activity to one of the 11 target proteins above, in a mutated strain of said bacterial cell. In this embodiment, PZA-sensitivity, e.g., binding or inhibitory activity, in the wild type version and lack of sensitivity, e.g., lack of binding and inhibitory activity, in the mutated version indicates PZA resistance.

In some embodiments, the target protein is obtained from a mutated version of a bacterial cell that is pyrazinamide (PZA) or Pyrazinoic Acid (POA) resistant. In certain aspects, for example, the target protein is normally PZA-sensitive in a wild type strain of the bacterial cell, but because of a mutation or other defect in the sequence of that target protein, it becomes PZA-insensitive and resistant. The test agent may be sensitive to the target protein and may bind or inhibit its activity even though PZA would not. For example, the wild type version may be M. tuberculosis, e.g., H37Rv, and the mutated strain may be DHM444.

In other embodiments, a method is provided for screening for a second antibacterial agent for enhancing or synergizing the activity of the potential antibacterial agent, pyrazinamide (PZA) or Pyrazinoic Acid (POA). For this aspect, the method involves (a) contacting a second test agent with a composition comprising a second target protein; and (b) determining whether the second test agent binds to, or inhibits activity of, the second target protein. In embodiments, binding of the second target protein or inhibition of the second target protein activity is indicative of a second potential antibacterial agent for enhancing or synergizing the activity of the potential antibacterial agent, pyrazinamide (PZA) or Pyrazinoic Acid (POA).

Examples of a second target protein, include but are not limited to a protein encoded by nuoH, NADH dehydrogenase, protein encoded by nuoN, NADH dehydrogenase, protein encoded by fdhF, formate dehydrogenase α-subunit, protein encoded by narH, nitrate reductase β-subunit, protein encoded by pncB1, nicotinatephosphoribosyltransferase, protein encoded by yjcE, Na/H exchanger, or protein encoded by kdpA, potassium transporting ATPase.

Structurally, PZA is an analog of nicotinamide. Mutation in pncA encoding PZase is the major mechanism for PZA resistance in M. tuberculosis. PZA is a prodrug, which requires activation to its active form POA by PZase enzyme of susceptible M. tuberculosis. PZA is believed to enter Mtb by passive diffusion, where it is converted to POA by the PZase. POA is an acid with a pKa of 2.9 and is therefore trapped within the cell as the carboxylate anion where it is possibly excreted by a weak efflux pump and passive diffusion. Protonated POA (HPOA) is reabsorbed into M. tuberculosis under acid conditions and accumulates inside the bacilli because the efflux pump is inefficient, causing cellular damage. The HPOA brings protons into the cell and could eventually cause cytoplasmic acidification such that vital enzymes will be inhibited. Small amounts of protonated POA capable of diffusion across the membrane have been proposed to collapse the proton gradient, reducing membrane potential and affecting membrane transport. At neutral or alkaline pH, POA does not get back in the cell, since over 99.9% of extracellular POA will be in charged anion form, which does not get into cells easily. This explains why PZA is active only at acidic pH but not active at neutral pH and also explains the correlation between MIC of PZA and acidic pH values using Henderson-Hasselbalch equation.

Energy production defect either due to chemical energy inhibitors or genetic mutations will lead to increased susceptibility to PZA. Also, M. tuberculosis mutants with defects in energy production show higher PZA susceptibility (MIC=10 μg/ml) than wild type strain (MIC=25-50 μg/ml) (Table 1 below).

TABLE 1

| Strain | Gene/Function | MIC (pH 5.5) |
|---|---|---|
| CDC1551 | Wild type | 25-50 (μg/ml) |
| MT3240 | nuoH, NADH dehydrogenase | 10 |
| MT3246 | nuoN, NADH dehydrogenase | 10 |
| MT2968 | fdhF, formate dehydrogenase α-subunit | 10 |
| MT1199 | narH, nitrate reductase β-subunit | 10 |
| MT1372 | pncB1, nicotinatephosphoribosyltransferase | 10 |
| MT2345 | yjcE, Na/H exchanger | 10 |
| MT1058 | kdpA, potassium transporting ATPase | 10 |
| MT48 | Transcription regulator MarR family | 25-50 |
| MT3006 | ATP-binding protein | 25-50 |
| MT3981 | Putative ATPase | 25-50 |

These mutants include mutations in NADH dehydrogenase subunits H and N (nuoH, nuoN), nitrate reductase narH, and formate dehydrogenase fdhF, and kdpA and yjcE involved in potassium and sodium ion transport, and pncB1 (Rv1330c) involved in NAD recycling. Nitrate reductase mutant, narH, and formate dehydrogenase mutant, fdhF, both involved in energy production under anaerobic conditions, are highly susceptible to PZA with a 5 fold reduction in MIC from 50 μg/ml in the wild type strain to 10 μg/ml in the mutants.

The pncB1 mutant, involved in NAD recycling, is also more susceptible to PZA. However, mutations in MT48 (marR), MT3006 (ATP binding protein), MT3981 (putative ATPase) did not have significant effect on PZA susceptibility. This confirms that energy production and NAD pathways are important for PZA action. Since POA disrupts membrane energy, any defect in the energy production pathways may enhance or synergize with PZA activity. Genes whose inactivation lead to increased or higher PZA sensitivity or cause increased synergy with PZA can serve as drug targets to develop new agents that synergize with PZA.

In yet further embodiments, a method is provided for amelioration or treatment of a disease or infection with bacteria to decrease persister formation and/or increase killing of a bacterial cell by administration of the potential antibacterial agent selected from the above screening methods. The agent may be used to decrease persister infection or formation, eliminate or reduce bacterial or pathogen infection or disease and/or increase killing of a bacterial cell.

As used herein “amelioration” or “treatment” is understood as meaning to lessen or decrease the signs, symptoms, indications, or effects of a specific disease. For example, amelioration or treatment of a bacterial infection can include a reduction in bacterial load, especially reduction in persister bacterial load. As used herein, “prevention” is understood as to limit, reduce the rate or degree of onset, or inhibit the development of a disease or condition. Prevention can include maintaining a subject with a bacterial load less than can be detected, or less than can manifest signs or symptoms in a subject, or prevention of relapse. Prevention, amelioration, and treatment can be a continuum and need not be viewed as discrete activities. Prevention, amelioration, and treatment can be effected by one or more doses of an agent of the invention.

In particular embodiments, the agent may be used alone or in combination with one or more of other antibacterial drugs for treating an individual for a bacterial disease or infection. The individual may be a human or non-human mammal. As used herein, the terms “subject”, patient”, and “individual” are used interchangeably.

For certain aspects, the agent and one or more of the antibacterial drugs may be administered prior to infection or after infection, or post-treatment to treat persistence or relapse. An antibacterial drug may be selected from one or more of isoniazid, ethambutol, rifampin and other rifamycins (rifapentine, rifabutin etc), aminoglycoside antibiotics (streptomycin, amikacin, kanamycin) or capreomycin, PAS, ethionamide, cycloserine, clofazimin, and other antimycobacterial agents, tetracyclines, amoxicillin, penicillins, clarithromycin, metronidazole, omeprazole, tetracycline, bismuth, vacomycin, azithromycin, trimethoprim, nitrofurantoin, quinolones, or doxycycline. In a particular embodiment, antibacterial drug may be selected from one or more of isoniazid (INH), rifampin (RIF), and ethambutol (EMB).

An “antibiotic” or “antibacterial agent” as used herein is understood as a compound inhibits or abolishes the growth of micro-organisms, such as bacteria, including bacteriostatic agents. Antibiotics include, for example, beta-lactams or cephalosporins, daptomycin, aminoglycosides, macrolides-lincosamides-streptogramins, linezolid, tetracylcines and quinolones, sulfa drugs or sulfonamides, piperazine, pyrantel pamoate. Beta-lactams include, for example the penicillins, cephalosporins, carbapenems and monobactams. Aminoglycosides include, for example, streptomycin, gentamicin, and neomycin. Macrolides, for example, azithromycin, clarithromycin, dirithromycin, erythromycin, roxithromycin, carbomycin A, josamycin, kitasamycin, midecamicine/midecamicine acetate, oleandomycin, spiramycin, troleandomycin, and tylosin/tylocine. Lincosamides include, for example, lincomycin and clindamycin. Streptogramins include, for example, pristinamycin and quinupristin/dalfopristin. Tetracyclines include, for example tetracycline, chlortetracycline, oxytetracycline, demeclocycline, doxycycline, lymecycline, meclocycline, methacycline, minocycline, rolitetracycline, tigecycline and glycylcycline antibiotics. Quinolones include, for example, cinoxacin, flumequine, nalidixic acid, oxolinic acid, piromidic acid, pipemidic acid, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin Mesilate, sparfloxacin, temafloxacin, tosufloxacin, clinafloxacin, gemifloxacin, moxifloxacin, gatifloxacin, sitafloxacin, trovafloxacin, ecinofloxacin, and prulifloxacin. Sulfa drugs or sulfonamides include, for example, acetazolamide, benzolamide, bumetanide, celecoxib, chlorthalidone, clopamide, dichlorphenamide, ethoxzolamide, indapamide, mafenide, mefruside, metolazone, probenecid, sulfacetamide, sulfadimethoxine, sulfanilamides, sulfamethoxazole, sulfasalazine, sultiame, sumatriptan, and xipamide.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

In another embodiment, a composition is provided comprising a pharmaceutically acceptable antibacterial agent in combination with a pharmaceutically acceptable agent identified from the above mentioned screening methods.

Thus, in connection with the administration of a drug or a combination of drugs, a drug or combination of drugs which are “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

A combination of two or more agents can be prepared or provided in an effective dose. The combination of two drugs can be provided in as a mixed formulation (e.g., prepared for administration as a single dose as a single tablet, capsule, or vial) or packaged for co-administration (e.g., in a single blister pack, or otherwise packaged together). A combination of agents need not be administered simultaneously. It is understood that different compounds have different pharmacokinetic and pharmacodynamic properties which may suggest dosing on different schedules to maintain an effective dose of each of the agents. It is understood that an effective dose of the combination of agents may be in an amount that is less than the effective dose of one or both of the agents alone.

In other embodiments, a diagnostic method is provided comprising the steps of detecting and/or measuring the level of a target protein in a biological sample wherein the presence and/or level of said target protein is correlated with a diagnosis, prognosis or treatment outcome. The diagnosis, prognosis or treatment outcome may be for a bacterial infection.

An assay as used herein refers to a procedure for testing and/or measuring the activity of a test agent, substance, compound, drug, cell, enzyme protein or other biological component or biochemical in an organism or organic sample. There are a number of techniques that can be used for performing the herein mentioned screening methods, or measuring metabolic activity or binding or cross-linkage, e.g., molecular weight assay, antibody assay, molecular sieving assay, mass spectrometry, or other assays. A number of laboratory assays can be used to identify the test agent, insensitivity or resistance, e.g., liquid or solid media growth assay, enzyme-linked immunosorbent assay (ELISA) or ELISA-based assays or using gas-liquid chromatography, mass spectrometry, PCR and sequencing, or other methods may be used.

In an exemplary embodiment, the target protein may be coupled to a solid support such as gel column or beads coated with target proteins to generate an affinity chromatographic matrix, target enzyme assays, trans-translation assay that relies on antibody to react with the tags produced from trans-translation, for screening the test agent. For comparison and confirmation, a control column may be utilized with a solid support that is coupled with ethanolamine without the target protein. A test solution is then prepared containing the test agent, added and a sample is run and washed with buffer to minimize nonspecific binding of proteins. A sample containing test agent bound to target protein may be eluted by ethylene glycol and the fractions are run on SDS-PAGE gels and stained. The test agent may then be excised and subjected to in-gel digestion with trypsin followed by analysis by Ion Trap Tandem Mass Spectrometry to determine the identity of the test agent.

A “cell free system” as used herein is a cell lysate that may or may not be fractionated. A cell free system can include purified proteins and nucleic acids.

As used herein, “changed as compared to a control reference sample” is understood as having a level of the analyte (e.g., colony forming unit) or activity (e.g., kinase activity, phosphatase activity) to be detected at a level that is statistically different than a sample from a normal, untreated, or control sample. Methods to select and test control samples were within the ability of those in the art. Depending on the method used for detection the amount and measurement of the change can vary. For example, a change in the amount of phosphorylation or dephosphorylation of analyte present will depend on the exact reaction conditions and the amount of time after exposure to the agent the sample is collected. Determination of statistical significance is within the ability of those skilled in the art.

As used herein, “colony forming unit” or “CFU” is understood as bacteria capable of resulting in the growth of a single colony on a bacterial culture plate.

“Contacting a cell” or “contacting a bacterial cell” is understood herein as providing an agent to a bacterial cell, in culture or in an animal, such that the agent can interact with the surface of the cell, potentially be taken up by the cell, and have an effect on the cell. The agent can be delivered to the cell directly (e.g., by addition of the agent to culture medium or by application, e.g., topical application to an infected area), or by delivery to the organism by an enteral or parenteral route of administration for delivery to the cell by circulation, lymphatic, or other means.

As used herein, “detecting”, “detection” and the like are understood that an assay performed for identification of a specific analyte in a sample, or a product from a reporter construct in a sample. The amount of analyte detected in the sample can be none or below the level of detection of the assay or method.

“Identify” or “identification” or the like as used herein as in “identification of an agent” is understood as characterization of a specific agent to determine specific characteristics of the agent to allow for determination of the chemical structures or properties of the agent. Identification can be accomplished by correlating the position, for example in the 96-well or 384-well plate to which the agent was added, or by determination of the chemical structure of an agent derived from a combinatorial chemistry library by NMR or other structural analysis, or by use of a radiofrequency tag or other identifying tag on the compound.

“Isoform” is understood herein as any of two or more functionally similar proteins that have a similar but not an identical amino acid sequence.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in a heterologous system). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. “Isolated” when used in reference to a cell means the cell is in culture (i.e., not in an animal). Isolated cells can be further modified to include reporter constructs or be treated with various stimuli to modulate expression of a gene of interest.

As used herein “killing assay” is understood as an experiment to determine a change in the amount of viable bacteria or CFU per volume of bacteria (e.g., per ml) over time in response to exposing or contacting the bacteria with one or more agents sequentially and/or simultaneously. Cells can be at any phase of growth or in non-growing persister or dormant state during the assay. Assays can be performed at any temperature but typically at 37° C., with or without shaking in the case of liquid culture, or after exposure to the agents the viability of bacteria can be assessed by subculture in liquid medium or on solid medium.

An in vitro cell-free trans-translation assay or kit with M. tuberculosis ribosome with or other ribosomes with M. tuberculosis RpsA and other components of trans-translation such as SmpB and tmRNA (SsrA) can be developed for screening compounds that inhibit trans-translation process. The detection could be in the form of antibody detection of the peptide tag encoded by tmRNA in the trans-translation system. A compound that inhibits trans-translation would be exhibited by absence of antibody reaction due to lack of production of the peptide tag as a result of inhibition by a particular compound.

Kits with one or multiple components of the assay, are included in the present invention. Such kits, in addition to the containers containing the multiple or unit doses of the assay, optionally include an informational package insert with instructions describing the use and attendant benefits of the assay components. In the kit the reagents can be provided in packaged combination in the same or separate containers, depending on the cross-reactivity and stability of the reagents, so that the ratio of reagents provides for substantial optimization of a signal from the reporter molecule used in the detection system. The diagnostic kit can comprise in packaged combination one or more test cartridges comprising a capillary and membrane. The kit may also include other reagents as may be employed in the tests.

The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

The phrase “library of compounds” or “compound library” is understood as a plurality of chemical compounds that may or may not be related by one or more property, such as activity, e.g., kinase inhibitor, phosphatase inhibitor, metal chelator; structure, e.g., peptides, nucleic acids including antisense nucleic acids, carbohydrates, antibodies; products of combinatorial chemistry; or by approval status, e.g., FDA approved compounds for administration to humans. Groups of compounds with no obvious relation can also be considered a library.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. USA 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

“Obtaining” is understood herein as manufacturing, purchasing, or otherwise acquiring.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

Homology/similarity and identity can be readily determined using any of a number of publicly available sequence alignment tools including, but not limited to, BLAST (Basic Local Alignment Sequence Tool) available from the National Center for Biotechnology (NCBI) website at http://www.ncbi.nlm.nih.gov/blast/Blast.cgi or using ClustalW at the European Biology Labs (EBL) website at http://www.ebi.ac.uk/Tools/clustalw/index.html. Other tools to determine sequence homology and identity can be found at, for example, http://restools.sdsc.edu/biotools/biotoolsl.html. Sequence homology can also be determined by methods known to those in the art (e.g., see Taylor W R. J Mol. Biol. 88:233-258, 1986).

As used herein, “plurality” is understood to mean more than one. For example, a plurality refers to at least two, three, four, five, one hundred, one thousand, or more.

“Reporter construct” as used herein is understood to be an exogenously inserted gene, often present on a plasmid, with a detectable gene sequence, under the control of a promoter sequence. The activity of the promoter is modulated upon binding of an agent that modulates transcription. Preferably, the gene product is easily detectable using a quantitative method. Common reporter genes include luciferase, beta-galactosidase, and green fluorescent protein (GFP). The reporter construct can be transiently inserted into the cell by transfection or transformation. Alternatively, stable cell lines or bacterial strains can be made by recombination using methods well known to those skilled in the art.

A “sample” as used herein refers to a biological material that is isolated from its environment (e.g., blood or tissue from an animal; cells or conditioned media from a culture) and is suspected of, or contains an analyte, such as a product from a reporter construct or an active kinase or phosphatase. A sample can also be a partially purified fraction of a tissue or bodily fluid. A reference sample can be a “normal” sample, from wild type bacteria or an uninfected subject or culture. A reference sample can also be from an untreated, but infected, subject sample or culture media; not treated with an active agent (e.g., no treatment or administration of vehicle only) and/or stimulus. A reference sample can also be taken at a “zero time point” prior to contacting the cell, culture, or subject with the agent to be tested.

A “subject” as used herein refers to living organisms. In certain embodiments, the living organism is an animal. In certain preferred embodiments, the subject is a mammal. In certain embodiments, the subject is a domesticated mammal. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of characteristics of the disease, condition, or syndrome such that a competent individual would diagnose or suspect that the subject was suffering from the disease, condition, or syndrome. Methods for identification of subjects suffering from or suspected of suffering from conditions such as bacterial infection is within the ability of those in the art. Subjects suffering from, and suspected of suffering from, a specific disease, condition, or syndrome are not necessarily two distinct groups.

“Therapeutically effective amount,” as used herein refers to an amount of an agent which is effective, upon single or multiple dose administration to the cell or subject, in prolonging the survivability of the subject or patient with such a disorder beyond that expected in the absence of such treatment.

An agent can be administered to a subject, either alone or in combination with one or more therapeutic agents, as a pharmaceutical composition in mixture with conventional excipient, e.g., pharmaceutically acceptable carrier.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1980). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the singular forms “a”, “an”, and “the” include plural forms unless the context clearly dictates otherwise. Thus, for example, reference to “an assay” includes a plurality of such assay.

Unless specifically stated or obvious from context, as used herein, the term “or” is understood to be inclusive.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Persisters are known to be tolerant to multiple antibiotics and stresses. It should be emphasized that “persisters” are relative and should be defined by highly specific conditions such as the type of antibiotics, antibiotic concentrations, the length of antibiotic exposure, the culture media and the growth phase. “Persisters” are not homogeneous, and consist of different bacterial subpopulations that are defined by specific conditions and times as discussed herein.

A persister or bacterial infection may include one or more of latent infections, chronic and recurrent infections, and biofilm infections. A disease can be one or more of Tuberculosis, Lyme disease, Syphilis, Peptic ulcer, Bacteremia/Sepsis, Endocarditis, Otitis media, Urinary tract infections, Brucellosis, and Biofilm infections. In certain aspects, a bacteria or pathogen can be one or more of M. tuberculosis, Borrelia burgdorferi, Treponema pallidum, H. pylori, S. aureus, Group A and Group B Streptococcus, Staphylococcus, enterococcus, S. pneumoniae, H. Influenzae, Moraxella catarrhalis, E. coli, Streptococcus, Chlamydia, Mycoplasma, M. genitalium, and Brucella abortus. In a particular embodiment, the bacterial infection is infection by Mycobacterium tuberculosis (Mtb).

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, patent applications, and journal articles are incorporated herein by reference in their entireties including the references cited therein, which are also incorporated herein by reference.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

This application claims priority to U.S. provisional application Nos. 61/355,786, filed Jun. 17, 2010, and 61/490,295, filed May 26, 2011, which are hereby incorporated herein by reference in their entirety.

### EXAMPLES

To identify potential targets that bind to POA (2-pyrazinecarboxylic acid) in Mtb, the Examples below were conducted using a proteomic approach to search for proteins that bind to POA by affinity chromatography.

### Example 1

### Synthesis of Pyrazinoic Acid (POA) Derivatives and Affinity Chromatography Column Preparation

POA derivative 5-hydroxyl-2-pyrazinecarboxylic acid or 6-hydroxyl-2-pyrazinecarboxylic acid was synthesized and purified by HPLC. The POA derivative (5-hydroxyl-2-pyrazinecarboxylic acid) was coupled to the epoxy-activated Sepharose 6B gel (GE Healthcare Life Science) to generate an affinity chromatographic matrix according to the manufacturer's instructions.

Suitable amounts (5 g) of epoxy-activated Sepharose 6B powders were allowed to swell in distilled water. After washing with distilled water on a glass filter, the sepharose was mixed in 50 ml of coupling buffer (0.1 M Na₂CO₃/NaHCO3 and 20% DMF, pH 13) containing 400 mg of 5-hydroxyl-2-pyrazinecarboxylic acid at 37° C. overnight. Then the column was washed to remove excess unbound ligand using coupling buffer. Any remaining active groups were blocked by 1M ethanolamine (pH 8.0) overnight at room temperature.

The control column was coupled with 1M ethanolamine only without POA derivative. The column was washed thoroughly with at least three cycles of alternating pH. Each cycle consisted of a wash with 0.1 M acetate buffer (pH 4.0) containing 0.5 M NaCl followed by a wash with 0.1 M Tris-HCl buffer pH 8 containing 0.5 M NaCl.

### Example 2

### Protein Lysate Preparation

M. tuberculosis H37Ra was cultured in Sauton's medium at 37° C. with gentle shaking for three weeks. Sauton's medium consisted of the following composition (per liter): 4 g of L-asparagine, 0.5 g of monopotassium phosphate, 0.5 g of magnesium sulfate, 50 mg of ferric ammonium citrate, 2 g of citric acid, 1 mg of zinc sulfate, and 60 ml of glycerol (with 0.05% Tween 80 added after sterilization). Mycobacterial cells were collected and washed with PBS buffer pH7.0 two times. Mycobacterial protein lysates were prepared by sonication followed by collecting the supernatants after centrifugation as described previously (Y. Zhang, R. Lathigra, T. Garbe, D. Catty, D. Young, Mol. Microbiol. 5, 381 (February, 1991)). The protein concentration of the lysates was determined by the Bradford method using BCA Protein Assay Kit (Pierce).

### Example 3

### Isolation of POA-Binding Proteins

The column was equilibrated with PBS buffer pH7.0. M. tuberculosis lysates (596 mg total) were loaded onto the POA-linked column (A) and control column (B), respectively. The lysates were passed through the affinity resin and the samples from both the POA column and the control column were run for at least 2 hours. Then the columns were washed with PBS buffer to minimize nonspecific binding of proteins until the baseline was stable. The samples were eluted by 25% ethylene glycol which changed the polarity and released the target proteins. The fractions (lysate, flow-through, washings, and elution) were run on 12.5% SDS-PAGE gels, followed by staining with Coomassie blue.

### Example 4

### Identification of POA Binding Proteins

The target proteins eluted from the POA column and separated on the SDS-PAGE gel were excised and subjected to in-gel digestion with trypsin followed by analysis by Ion Trap Tandem Mass Spectrometry to determine the identity of the POA binding proteins.

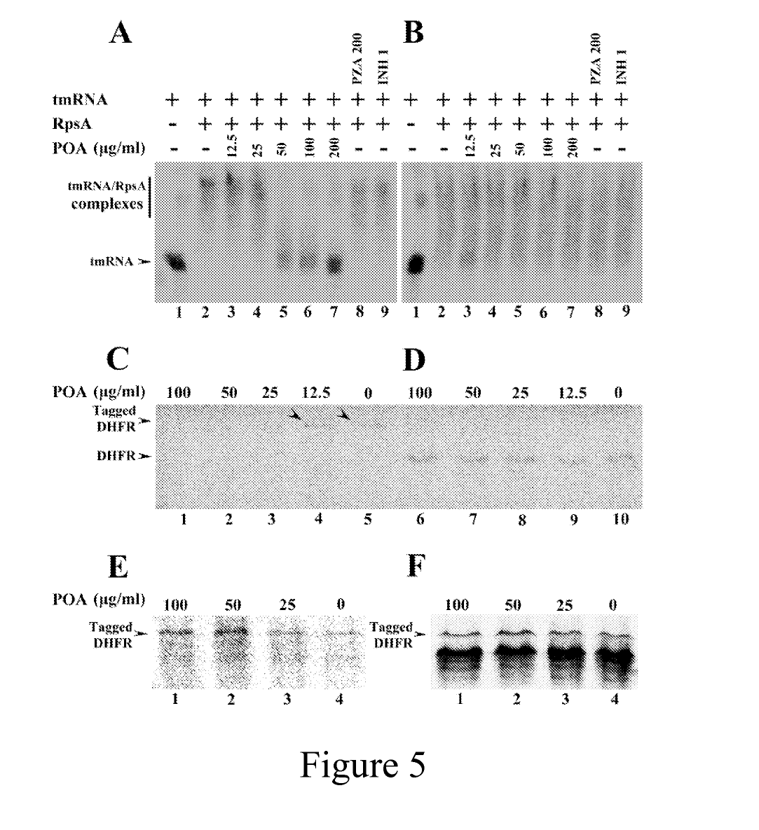

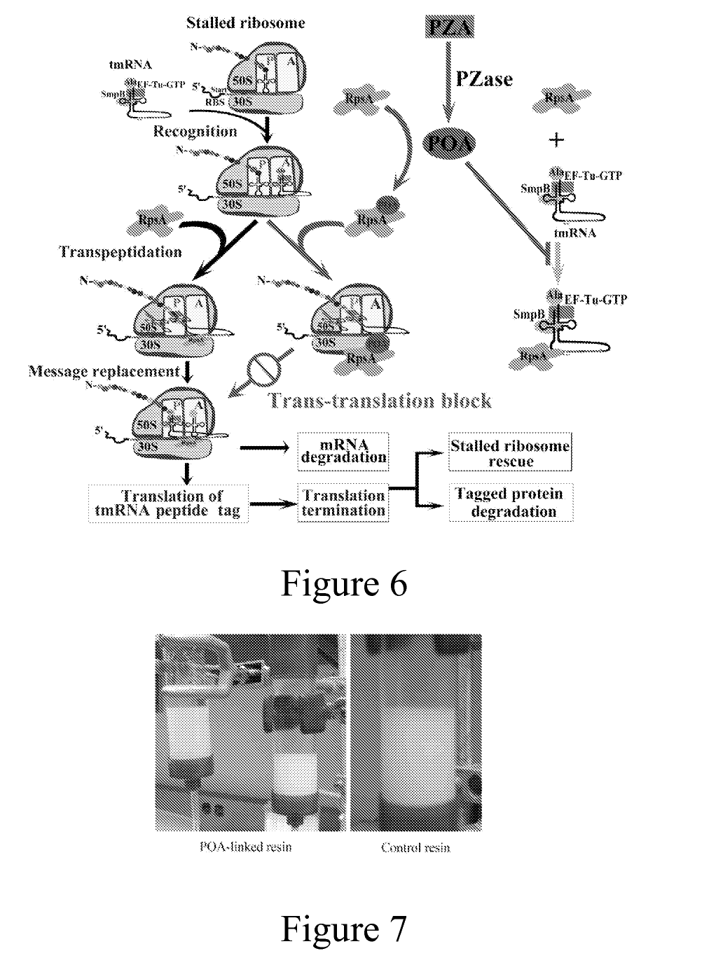

Binding studies with M. tuberculosis cell lysates revealed several proteins that bound to POA (FIG. 1). FIG. 5A shows the results from the POA analog 5-hydroxyl-2-pyrazinecarboxylic acid, which was synthesized and covalently coupled to Epoxy Sepharose 6B column. FIG. 5B shows the results of the control—ethanolamine, which was also coupled to a separate column. In contrast, no proteins bound to the control column, indicating that the proteins bound specifically to POA.

### Example 5

### RpsA, the Largest 30S Ribosomal Protein S1 (Rv1630)

Mass spectrometry analysis and subsequent database searches identified the major POA binding protein as RpsA (Tables 2 and 3), the largest 30S ribosomal protein S1 (Rv1630) from Mtb.

TABLE 2
RpsA identified using Ion Trap
Tandem Mass Spectrometry.

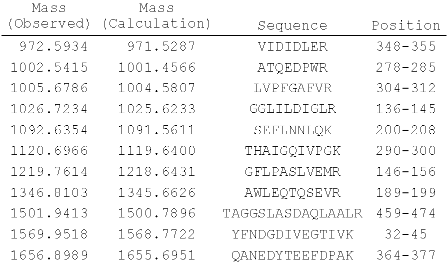

TABLE 3

| Example # | H37Rv Gene | Locus Tag | Function | Size |
|---|---|---|---|---|
| Ex 6 | rpsA | Rv1630 | 30S ribosomal | 481aa |
| | | | protein S1 | |

### Methods and Materials

### Cloning, Sequencing, Expression and Purification of M. Tuberculosis RpsA

The rpsA encoding the M. tuberculosis ribosomal protein S1 was amplified by PCR from M. tuberculosis H37Rv and DHM444 (A. Scorpio et al., Antimicrob. Agents Chemother. 41, 540 (Mar, 1997)). genomic DNA using a forward primer 5′-ATAGGATCCATGCCGAGTCCCACCGTCAC-3′ (SEQ. ID NO: 1) containing a BamHI restriction site and a reverse primer 5′-GACAAGCTTTCAAGCGCTGCCGGCGAGTT-3′(SEQ. ID NO: 2) with a HindIII restriction site. The DHM444 rpsA was subjected to DNA sequencing and was found to contain an amino acid alanine deletion at the C-terminus of the RpsA. The resulting DNA fragments were digested with BamHI and HindIII, and ligated to plasmid pET-28a with the same enzyme digested to yield recombinant plasmids pETrpsA and pET rpsA444. The RpsA was overexpressed in E. coli strain BL21 (DE3) transformed with wild type RpsA construct pETrpsA or mutant RpsA construct pET rpsA444 and induced by IPTG (1 mM). The supernatant containing the recombinant RpsA was purified on Ni²⁺-NTA agarose (Qiagen). Immobilized recombinant proteins were washed by a 20-50 mM imidazole gradient and eluted with buffer (20 mM Tris-HCl, 300 mM NaCl, pH 8.0 and 200 mM imidazole). The two purified proteins were dialyzed against 10 mM Tris-HCl buffer (pH 7.5) to remove imidazole.

### Isothermal Titration Calorimetry (ITC) Binding Study

Titration of recombinant RpsAs from M. tuberculosis H37Rv, DHM444, and M. smegmatis with POA was performed in a VP-ITC titration calorimetric system (MicroCal, LLC) at 25° C. Initially, ITC was used to determine whether any interaction existed between RpsA of M. tuberculosis H37Rv and POA. Solutions of 10 μM wild type RpsA or mutant RpsA from DHM44 strain and M. smegmatis RpsA in 10 mM phosphate buffer (pH 7.4) containing 0.1% DMSO were titrated with 100 μM POA or PZA. Blank titration of drug solution in the same buffer in the absence of RpsA protein was performed. The next step was to further titrate with saturated POA concentration against 10 μM protein. In this step the binding constants were estimated from the obtained isotherms using the calorimetric analysis Origin software. Typically, 27 injections (10 μl per injection) were made at 300s intervals, and reaction temperature was at 25° C. The heat of reaction per injection (microcalories per second) was determined by integration of the peak areas.

### RpsA Overexpression in M. Tuberculosis

The rpsA gene from M. tuberculosis H37Rv was amplified by PCR using primer pair RpaF: 5′-CGCTCTAGACAACCGTCAAGTGCGGGAGG-3′ (SEQ. ID NO: 3) and RpaR: 5′-CGCTCTAGAGCTCCGTCTGCAAGCAGGAT-3′ (SEQ. ID NO: 4) (XbaI site underlined) and ligated into shuttle vector pOLYG and transformed into E. coli DH5. The rpsA recombinant plasmid along with vector control pOLYG was then electroporated into competent cells of M. tuberculosis H37Ra as described. Transformed M. tuberculosis cells were plated on 7H11 agar plates containing hygromycin (75 μg/ml). The transformants were grown in 7H9 medium plus ADC (bovine albumin-detrose-catalase) supplement containing hygromycin and used for drug susceptibility testing.

### Drug Susceptibility Testing

The MICs of PZA for different M. tuberculosis strains (wild type and wild type/pOLYG, and wild type/pOLYG+rpsA) were performed in 7H11 agar and 7H9 liquid medium at acid pH 5.5 as described (Y. Zhang, S. Permar, Z. Sun, J. Med. Microbiol. 51, 42 (Jan, 2002)). The MICs of the M. tuberculosis strains to control drugs isoniazid, rifampin, streptomycin, kanamycin and norfloxacin were determined similarly as for PZA except that the pH (pH6.8) of the media was not adjusted.

Synthesis and Purification of tmRNA

M. tuberculosis ssrA gene under the control of the T7 promoter was amplified by PCR from M. tuberculosis H37Rv genomic DNA with a primers 5′-TAATACGACTCACTATAGGATCTGACCGGGAAGTTAATGGC-3′ (SEQ. ID NO: 5) containing the T7 promoter sequence and 5′-GATCAGATCCGGACGATCGGCATCG-3′ (SEQ. ID NO: 6). The M. tuberculosis tmRNA was transcribed in vitro and purified according to manufacturer's protocol of TranscriptAid™ T7 High Yield Transcription Kit (Fermentas).

### Gel Shift Binding Assay

Binding reactions (20 μl) contained recombinant H37Rv RpsA or DHM444 RpsA, 80 μM tmRNA, 10 mM Tris-HCl (pH 7.5), 100 mM NH₄Cl, 10 mM MgAc, 1 mM DTT, 100 μg/ml BSA (New England Biolabs), 100 μg/ml E. coli tRNA (Roche), and 5% glycerol and were incubated at 4° C. for 45 min in the presence of 20 units of RNasin. tmRNA was treated before the binding reaction by heating at 80° C. for 2 min and then slowly cooled to room temperature for 20 min. The POA inhibition assay was investigated with 100 μg/ml POA in binding reaction. POA and recombinant H37Rv RpsA or DHM444 RpsA were incubated at 25° C. for 30 min. Samples were electrophoresed in a 5% native polyacrylamide gel pre-ran at 100 V for 1 hour in TGE buffer (25 mM Tris, 190 mM glycine, 1 mM EDTA and 2.5% glycerol), and then ran for 6-7 hr at 18 mA per gel at 4° C.

### Ribosome Purification

Bacterial ribosomes were prepared as described (G. Spedding, Ribosomes and Protein Synthesis, a Practical Approach. (IRL Press at Oxford University Press, Oxford, New York, 1990)). Briefly, log phase cells of M. tuberculosis H37Ra or M. smegmatis were resuspended in buffer A (20 mM Tris-HCl, pH 7.5 at 4° C., 10.5 mM magnesium acetate, 100 mM NH₄Cl, 0.5 mM EDTA, and 3 mM 2-mercaptoethanol) at a ratio of 1 gram wet weight of bacteria in 2.5 ml buffer A. The bacteria were ruptured in cold French press at between 15,000 and 18,000 psi at least 3 times. The ruptured cellular mixture was centrifuged at 30,000×g for 1 hr to remove whole cells, cellular debris, and cell walls. The top three-fourths supernatant was mixed with an equal volume of 1.1 M sucrose cushions made up in buffer A except 0.5 M NH₄Cl and centrifuged at 100,000×g for 15 hours to obtain the cell-free particulate fraction. The pellets were washed and suspended gently in 10 mM Tris-HCl (pH7.5), 60 mM NH₄Cl and 3 mM 2-mercaptoethanol, and then dialyzed to remove sucrose. Ribosomes and subunits were aliquoted and rapidly frozen in a dry ice-methanol bath prior to storage at −80° C. (1 A260 unit is equivalent to 23 pmol ribosomes when determined in 25 mM Tris-HCl, 10 mM Mg²⁺, and 100 mM NH₄Cl.

### In Vitro Translation and Trans-Translation Assays

The in vitro translation and trans-translation reactions were performed as described (T. Kanda, K. Takai, S. Yokoyama, H. Takaku, J. Biochem. 127, 37 (Jan, 2000)) with some modifications. PURExpress (NEB #E3313) was used for in vitro translation and trans-translation experiments. The purified M. tuberculosis H37Ra, M. smegmatis or E. coli ribosomes were used in the system. Plasmid with T7 promoter before the E. coli DHFR gene was used as positive control template DNA for translation from PURExpress kit and a DNA template with 8×AGG rare codons at the end of the DHFR gene was used for trans-translation. The template DNA fragment with 8×AGG codons for trans-translation was amplified by PCR with primers

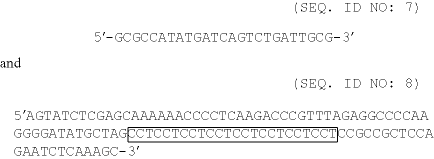

using positive control plasmid as template to replace the normal DHFR gene. The boxed region represents the 8 rare AGG codons as the recognition signal for trans-translation. ³⁵S-methionine was used for in vitro labeling for protein synthesis. POA, nicotinic acid, an analog of POA as a control, both dissolved in DMSO, were used at appropriate concentrations. The trans-translation reaction contained 0.3 μM tmRNA and 0.6 μM SmpB besides the M. tuberculosis, M. smegmatis or E. coli ribosomes and other components for translation. The reaction was incubated at 37° C. for 2 hours. The translated proteins were analyzed by 20% SDS-PAGE and visualized using phosphor imager FLA5000 (Fujifilm).

### Results, Analysis and Discussion

Target overexpression often confers increased drug resistance. To confirm that RpsA was indeed a target of PZA, we overexpressed the wild type RpsA in Mtb and measured the PZA sensitivity of these bacilli compared to bacilli carrying only the empty vector control. Overexpression of RpsA caused a 5-fold increase in the minimum inhibitory concentration (MIC) of PZA (MIC=500 μg/ml) compared with the vector control and the parental Mtb strain (MIC=100 μg/ml) at pH 5.5. The susceptibility of the RpsA overexpressing Mtb strain to other drugs, including isoniazid, rifampin, streptomycin, kanamycin, and norfloxacin, remained the same as the parent strain or the vector control strain.

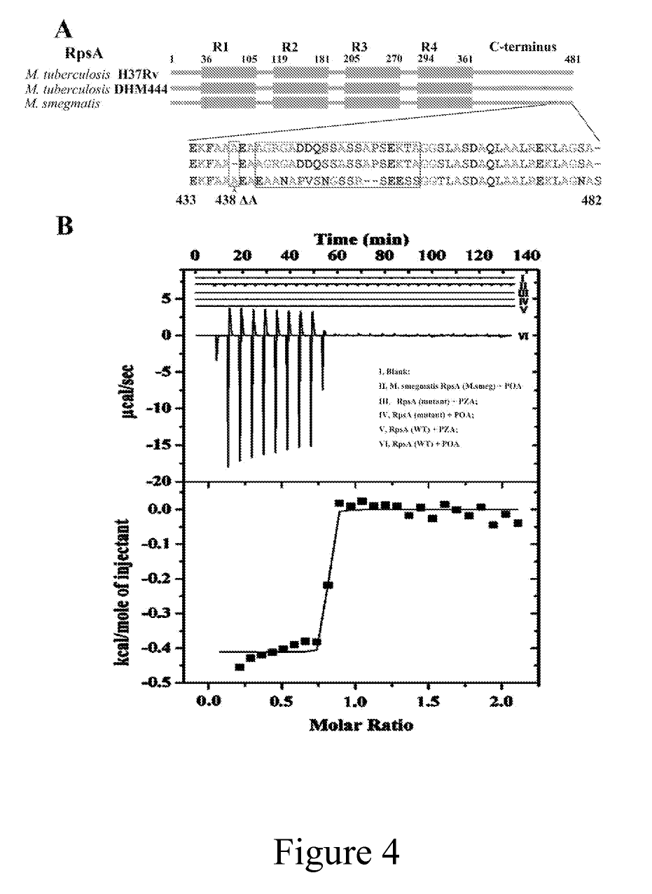

Most PZA-resistant M. tuberculosis strains have mutations in pncA that prevent conversion of PZA to POA. A small number of PZA-resistant strains, however, have been reported that do not have pncA mutations. We previously identified a low level PZA-resistant Mtb clinical isolate DHM444 (MIC=200-300 μg/ml PZA compared with 100 μg/ml in the sensitive control strain H37Rv) that lacked any pncA mutations. This suggested that its resistance could be due to alterations in RpsA. We therefore sequenced the rpsA gene from this strain and found that it contained a 3-bp GCC deletion at the nucleotide position 1314 resulting in deletion of an alanine at amino acid 438 (AA438) in the C-terminus of RpsA (FIG. 4A), a region that is not considered to be strictly required for protein synthesis in vivo.

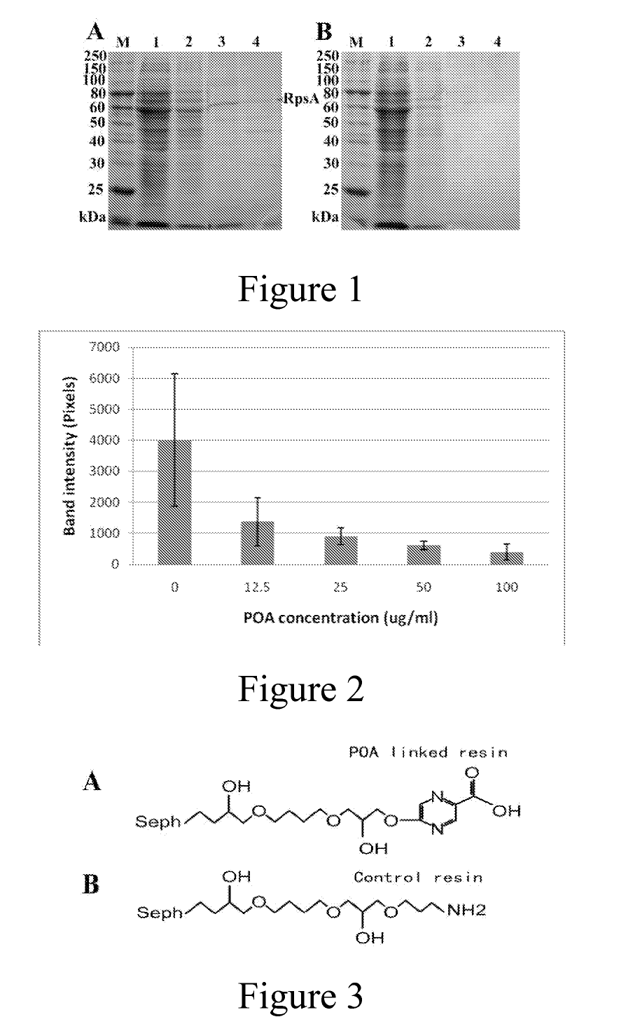

To determine if the mutant RpsA from the PZA-resistant strain DHM444 has any defect in POA binding, we overexpressed and purified the mutant RpsA (designated RpsA_{ΔA438}), the wild type M. tuberculosis RpsA, and the M. smegmatis RpsA, and assessed their ability to bind to POA using isothermal titration calorimetry (ITC). The wild type Mtb RpsA was found to specifically bind to POA (FIG. 4B, VI) with K=(7.53±2.21)×10⁶ M⁻¹, ΔH=−410.9±8.693 Kcal·mol⁻¹, ΔS=27.6 cal·mol⁻¹·K⁻¹ (FIG. 4B, lower panel), but not to the prodrug PZA as expected (FIG. 4B, V). However, the mutant RpsA_{ΔA438} from the PZA-resistant strain DHM444 failed to bind to POA or PZA (FIG. 2B, IV, III), while the RpsA from naturally PZA-resistant M. smegmatis bound to POA only very weakly (FIG. 4B, II). Since the mutant Mtb DHM444 RpsA and the M. smegmatis RpsA had little or no binding to POA (FIG. 4B), it can be inferred that POA binds to the C-terminus of the wild type M. tuberculosis RpsA (FIG. 4A). From the protein sequence alignment of RpsA from different mycobacterial species, the C-terminal region, where the mutation occurs in the PZA-resistant M. tuberculosis strain DHM444, is also the most variable region in the PZA sensitive versus resistant mycobacterial species (FIG. 4A), indicating that changes in this region may alter PZA susceptibility.

Without being bound to any theory, it is believed that the ribosomal protein 51 (RpsA) is essential for translation, binding directly to both the ribosome and upstream sequences of mRNA. In addition to its function in translation, the C-terminus of the RpsA is involved in trans-translation by specifically binding to tmRNA. Trans-translation is a process involved in rescuing ribosomes that have stalled while in the process of decoding mRNA, tagging the truncated proteins for degradation. Trans-translation has been associated with stress survival, virulence, and recovery from nutrient starvation. We therefore evaluated whether the mutant RpsA has any deficiency in tmRNA binding compared with the wild type Mtb RpsA and whether POA affects the interaction between RpsA and tmRNA. Specific binding of RpsA to tmRNA was assessed by changes in gel mobility in the presence of excess tmRNA. The wild type Mtb RpsA and the mutant RpsA_{ΔA438} both bound to the tmRNA in the absence of POA (FIG. 5A, 5B) although the mutant appeared to bind more weakly overall (FIG. 5B). However, when POA was added to the system, it inhibited the wild type Mtb RpsA from binding to tmRNA at its MIC concentration of 50 μg/ml (FIG. 5A), but did not inhibit binding of the mutant RpsA_{ΔA438} (FIG. 5B). The prodrug PZA and the control drug INH had no effect on the binding of wild type or mutant RpsA to the tmRNA (FIG. 5B).

RpsA is known to bind to the 3′ terminus of the mRNA-like portion of the tmRNA, which has been shown to form a multimeric complex with SmpB, EF-Tu and RpsA for enhanced efficiency of trans-translation. Since POA bound to Mtb RpsA (FIG. 4B), and since RpsA is involved in both translation and trans-translation, we tested whether POA inhibited the translation or the trans-translation function of RpsA. POA had no effect on conventional protein synthesis (FIG. 5D). To examine trans-translation we utilized an in vitro cell-free translation system using the target gene coding for dihydrofolate reductase (DHFR) containing ribosomes from Mtb, M. smegmatis, or E. coli. The DHFR template DNA contained a T7 promoter for transcription and a ribosome-binding site before the DHFR gene with a stop codon or a rare codon cluster for stalled ribosome formation for assessing trans-translation. In the presence of a rare codon cluster in the target DHFR gene, ribosomes stall and translation is blocked. When recombinant Mtb SmpB and in vitro transcribed Mtb tmRNA were added a higher molecular weight protein with the tmRNA derived peptide tag was observed for message carrying the rare codon cluster (FIG. 5C, Lane 5), but no such band was observed for the no template control reaction (data not shown). POA inhibited the trans-translation of DHFR with the rare codon cluster at 25 μg/ml or more (FIG. 5C, Lane 3, Lane 2 and Lane 1) in a concentration dependent manner (FIG. 2). However, POA did not affect the translation of the normal DHFR gene even at 100 μg/ml with Mtb ribosomes (FIG. 5D) nor did it inhibit the trans-translation of the template bearing the rare codon cluster with either M. smegmatis or E. coli ribosomes (FIGS. 5E and 5F). These observations support that POA directly binds to RpsA to cause the inhibition of trans-translation. Under the same conditions, nicotinic acid (50 μg/ml), an analog of POA as a control compound, did not inhibit trans-translation (data not shown).

The Mtb RpsA protein consists of four imperfect repeats of the S1-like domain thought to function directly in binding of RNA, bridging the mRNA (or tmRNA) template and the head of the 30S ribosome terminated at the C-terminus by a 117 amino acid segment. The E. coli RpsA protein contains six repeating S1 domains, with the two N-terminal domains required for ribosome binding. The C-terminal domains have been shown to be specifically involved in trans-translation, thus the deletion of Ala438 observed in our clinical isolate is consistent with POA exerting an effect on ribosome rescue. The deletion occurs within a region homologous (35% identical) to the protein Xrcc4 involved in illegitimate DNA recombination that forms an extensive α-helix. Homology modeling of the region suggests that Ala438 lies several turns in to the α-helix connecting a small globular domain to a long helical stalk involved in dimerization. Intriguingly this region has been proposed to be the primary site of nucleic acid interaction in Xrcc4. There are numerous basic residues along the helical face and particularly in the short linker between this helix and the globular domain potentially involved in DNA binding particularly Arg423, Arg424, His 425 and Lys426 that may interact directly with POA disrupting the site of tmRNA interaction.

Trans-translation is dispensable during active growth conditions but becomes important for bacteria in managing stalled ribosomes or damaged mRNA and proteins under stress conditions. It is required for stress survival and pathogenesis in some bacteria. The levels of RpsA or S1 protein are known to correlate with growth rate, and stress conditions (stationary phase, starvation, acid pH, hypoxia) that halt bacterial growth down-regulate RpsA in bacteria. When POA binds to RpsA it prevents the binding of tmRNA to RpsA such that tmRNA cannot function to rescue stalled ribosomes. PZA inhibition of the trans-translation process may therefore be plausibly linked to an interference with survival under stressful, non-replicating conditions in Mtb. The finding that POA binds to RpsA and inhibits the trans-translation process helps to explain how diverse stress conditions such as starvation, acid pH, hypoxia, and energy inhibitors and other drugs could all potentiate PZA activity.

It is worth noting that the conditions that down-regulate RpsA are exactly the stress conditions that also potentiate PZA activity. That PZA activity is enhanced by diverse stress conditions has been a mystery. The identification of RpsA as a target of PZA has now afforded a plausible explanation. It is known that under many stress conditions, cells stop growing, translation slows, and levels of RpsA are decreased. Faced with the need to continue to produce proteins required for survival during non-replicating persistence, ribosome rescue becomes even more critical than during periods of rapid growth. POA could therefore saturate the lower levels of RpsA more efficiently and thus show higher activity in metabolically quiescent persister cells than in actively replicating cells.

### Example 6

### Materials and Methods

### Culture Condition and Protein Lysate Preparation.

M. tuberculosis strain H37Ra was obtained from ATCC and cultured in liquid Sauton's medium at 37° C. with gentle shaking for three weeks. Sauton medium consisted of the following composition (per liter): 4 g of L-asparagine, 0.5 g of monopotassium phosphate, 0.5 g of magnesium sulfate, 50 mg of ferric ammonium citrate, 2 g of citric acid, 1 mg of zinc sulfate, and 60 ml of glycerol (with 0.05% Tween 80 added after sterilization). Mycobacterial cells were collected and washed with PBS buffer pH7.0 two times. Mycobacterial protein lysates were prepared by sonication followed by collection the supernatants after centrifugation as described previously. The protein concentration of the lysates was determined by the Bradford method using BCA Protein Assay Kit (Pierce).

### Synthesis of Pyrazinoic Acid (POA) Derivative and Affinity Chromatograghy Column Preparation.

POA derivative 5-hydroxyl-2-pyrazinecarboxylic acid was synthesized and purified by HPLC. The POA derivative (5-hydroxyl-2-pyrazinecarboxylic acid) was coupled to the epoxy-activated Sepharose 6B gel (GE Healthcare Life Science) to generate an affinity chromatographic matrix according to the manufacturer's instructions. Briefly, the suitable amounts (5 g) of epoxy-activated Sepharose 6B powders were allowed to swell in distilled water. After washing with distilled water on a glass filter, the sepharose was being shaken in 50 ml of coupling buffer (0.1 M Na2CO3/NaHCO3 and 20% DMF, pH13) containing 400 mg of 5-hydroxyl-2-pyrazinecarboxylic acid in a stoppered vessel at 37° C. overnight. Then the column was washed to remove excess unbound ligand using coupling buffer. Any remaining active groups were blocked by 1M ethanolamine pH 8.0 overnight at room temperature. The control column was coupled with 1M ethanolamine only without POA derivative. The column was washed thoroughly with at least three cycles of alternating pH. Each cycle consisted of a wash with 0.1 M acetate buffer pH 4.0 containing 0.5 M NaCl followed by a wash with 0.1 M Tris-HCl buffer pH 8 containing 0.5 M NaCl.

### Isolation of POA-Binding Proteins.

The column was equilibrated with PBS buffer pH7.0. The M. tuberculosis lysates (596 mg total, i.e., 40 ml×14.9 mg/ml) were loaded onto the POA-linked column (A) and also control column (B), respectively. The columns were run for at least 2 hours. Then the columns were washed with PBS wash buffer to minimize nonspecific binding of proteins until the baseline was stable. The samples were then eluted by 25% ethylene glycol which changed the polarity and released the target proteins. The fractions (lysate, flow-through, washings, and elution) were run on 12.5% SDS-PAGE gels, followed by staining with Coomassie blue.

### Identification of POA Binding Proteins.

The different protein bands from SDS-PAGE gel were excised and subjected to analysis by Matrix Assisted Laser Desorption/Ionization Time-of-Flight (MALDI-TOF) Mass Spectrometry to identify the POA binding proteins.

### Example 7

### Overexpression and Purification of M. Tuberculosis Endonuclease IV

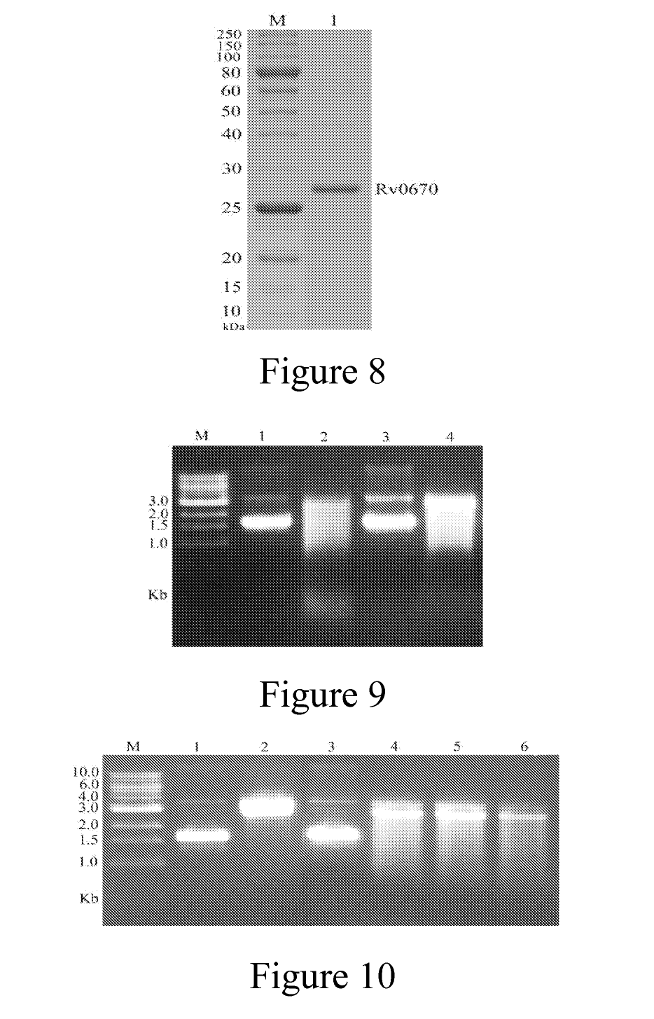

The M. tuberculosis Endo IV gene (Rv0670) was amplified by PCR from M. tuberculosis H37Rv genomic DNA using the following forward and reverse primers: EndF, 5′-GCGACATATGCTCATTGGTTCGCATGTCAG-3′ (SEQ. ID NO: 9) and EndR, 5′-GCGTAAGCTTCAGCTGCCGGTTCTTTCCC-3′ (SEQ. ID NO: 10), designed from the published genomic sequence. PCR amplification of the gene was performed (95° C. for 15 min, then 95° C. for 1 min 56° C. for 1 min, and 72° C. for 1-3 min for 25 cycles followed by 72° C. for 5 min) in a reaction volume of 100 μl containing 200 nM of each primer, 200 μM of each deoxynucleoside triphosphate, 100 ng of M. tuberculosis genomic DNA, and 2 units of hot-start DNA polymerase (Invitrogen). The amplified product of the anticipated size, 756 bp for the Endo IV (Rv0670) gene, was cloned in frame with a N-terminal His tag in the overexpression vector pETev (modified from pET28a) and transformed into E. coli BL21 cells according to the manufacturer's instructions. E. coli BL21 cells harboring the pETev/Mtb-Endo IV, were grown overnight at 37° C. in 1-Liter to an A600 of 0.5, at which point IPTG was added to a final concentration of 0.4 mM. The cells were allowed to grow for a further 4 h at 37° C. or for overnight at 16° C. and were then collected by centrifugation at 3000×g for 20 min at 4° C. The cells were resuspended in 20 ml of buffer (50 mM Tris-HCl buffer, pH 7.5, 300 mM NaCl, 20 mM imidazole) and lysed by sonication. The crude extract was spun by centrifugation a 12,000×g for 30 min at 4° C. The inclusion bodies (IB) containing the overexpressed protein were solubilized in 8 M urea and refolded using an on-column chemical refolding method. Briefly, solubilized IBs were bound to Ni-nitrilotriacetic acid resin (QIAGEN) and washed with buffer containing gradual urea. Elution was performed with buffer containing 50 mM Tris-HCl, pH 7.0, 300 mM NaCl, 5 mM β-mercaptoethanol, and 250 mM imidazole. The homogeneity of protein preparations was verified by SDS-PAGE. To exclude contamination of Endo IV by DNA glycosylases and non-specific nucleases, the enzyme prep was heat treated at 75° C. for 30 min. The Rv0670 (Endo IV) protein was overexpressed in E. coli BL21 and purified by His tag-nickel affinity chromatography. A single band of 27 kDa (Endo IV) was observed on SDS-PAGE (see FIG. 8).

TABLE 4

| Example # | H37Rv Gene | Locus Tag | Function | Size |
|---|---|---|---|---|
| Ex 7 | nfo (end) | Rv0670 | Endonuclease IV | 252 aa |
| | | | (DNA Repair) | |

Purified Rv0670 (Endonuclease IV) has Endonuclease V Activity that is Inhibited by POA

DNA cleavage by Rv0670 protein (0.5 ug) was assayed in 20 μl reaction mixtures at 37° C. in mixtures containing pUC19 plasmid DNA (1.5 ug) in a reaction buffer containing 20 mM Tris-HCl, pH7.5, 100 mM NaCl, and 1 mM DTT for 1 hr. After reaction, the reaction products were added with urea up to 7M and loading buffer, heated for 5 min at 95° C., and fractionated by electrophoresis on a denaturing 20% polyacrylamide gel containing 7M urea in TBE buffer. The gel was stained with fast blast DNA stain. When Rv0670 (Endo IV) was incubated with pUC19 plasmid DNA, Rv0670 digested the plasmid DNA and formed a smear (FIG. 9, Lane 2). The smear banding pattern was the same as that of E. coli endonuclease V digested pUC19 (FIG. 9, Lane 4), but not endonuclease IV from E. coli (FIG. 9, Lane 3). Rv0670 cut plasmid DNA with nonspecific site when several of the DNA fragments were sequenced.

When different drugs POA, PZA and INH (25 mM) were added to the above reaction system, only POA inhibited the activity of Rv0670 (Endo IV/V) completely because the banding pattern (FIG. 10, Lane 3 added with POA) was the same as that of the no enzyme Endo IV/V (Rv0670) control (FIG. 10, Lane 1), which moved faster due to supercoiling forms. For the control Endo IV/V reactions added with INH (25 mM), PZA (25 mM) and DMSO, the bands were almost the same as the drug free control, indicating no inhibition (FIG. 10, Lane 4, Lane 5 and Lane 6). This represents an exciting new finding and forms the basis for more detailed study of Endo IV/V (Rv0670) as a target of POA in this proposal (see Research Design). In addition, we found that POA seems to inhibit M. tuberculosis Endo IV/V irreversibly since dilution or dialysis could not relieve the inhibition of Endo IV/V.

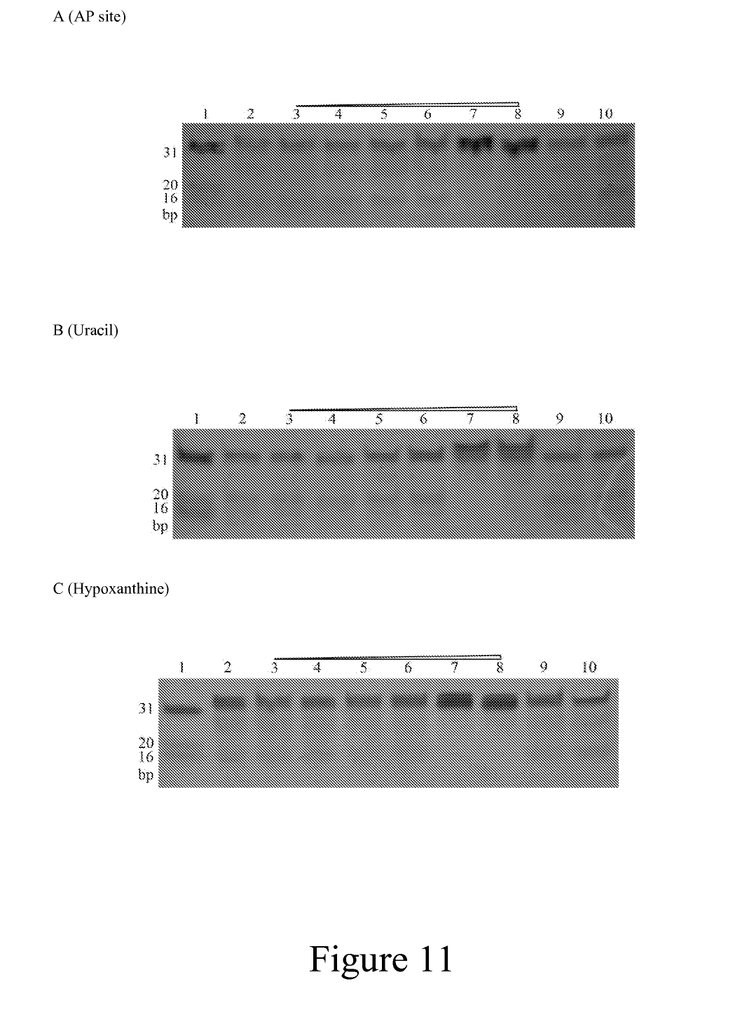

Since the above plasmid DNA cleavage experiment suggests that Rv0670 (Endo IV) behaved like Endo V of E. coli (FIG. 9), we therefore tested Rv0670 using common Endo V substrates, i.e., oligonucleotides containing different types of lesions apurinic/apyrimidinic (AP), uracil (U) or hypoxanthine (H) in reaction buffer containing 10 mM Tris-Cl, pH 7.5, 50 mM NaCl, 5 mM MgCl₂, and 1 mM DTT. The endonuclease activity of Rv0670 (0.5 ug) was assayed using a double stranded oligos (1 uM) 5′-CTGACTGCATA-X-GCATGTAGACGATGTGCAC-3′ (SEQ. ID NO: 11) and complement strand 5′-GTGCACATCGTCTACATGCTTATGCAGTCAG-3′ (SEQ. ID NO: 12) where X was nucleotide containing apurinic/apyrimidinic (AP), uracil (U) or hypoxanthine (H). Interestingly, Rv0670 recognized and cleaved the DNA lesions such as a basic site, uracil and hypoxanthine base, consistent with its activity as Endo V rather than Endo IV. Thus we renamed Rv0670 as Endo IV/V. POA inhibited Rv0670 activity specifically in a dose dependent manner (FIG. 11, Lanes 3 to 8, esp. Lane 7 and 8), whereas INH and PZA (as a prodrug) did not inhibit the endonuclease activity of Rv0670 even at 25 mM (FIG. 11, Lanes 9 and 10).

These studies clearly demonstrate that the purified Rv0670 has Endo V activities and that their activities can be specifically inhibited by POA but not by INH or the prodrug PZA before its activation.

### Examples 8-16

### Other Protein Targets of PZA/POA

POA analogues 5-hydroxyl-2-pyrazinecarboxylic acid and 6-hydroxyl-2-pyrazinecarboxylic acid have hydroxyl group were coupled to epoxy active groups on the Epoxy Sepharose 6B column at alkaline pH (pH9˜13) (see FIG. 7) as set forth in the previous the Examples. Binding studies were performed using the above POA-linked column and control affinity chromatography.

Bacterial lysates were passed through an affinity resin and the sample was eluted with 25% ethylene glycol. The eluants were separated by SDS-PAGE. Mass Spectrometry analysis was carried out. Several protein bands that bound to POA were seen on SDS-PAGE gel. Mass Spectrometry analysis identified 9 proteins, 5 ribosomal proteins (RpsD, RplJ, RplI, RplL, RpmC), 2 proteins with known function (HbhA for Iron-regulated heparin-binding hemagglutinin, and GpsI for polynucleotide phosphorylase), 1 putative DNA repair ATPase (Rv2731), and 1 protein with unknown function (Rv3169), that may be potential targets of POA (see Table 5).

TABLE 5
POA binds to multiple protein targets in M. tuberculosis

| Example # | H37Rv Gene | Locus Tag | Function | Size |
|---|---|---|---|---|
| Ex 8 | gpsI | Rv2783c | Polynucleotide | 752aa |
| | | | phosphorylase/ | |
| | | | polyadenylase | |
| Ex 9 | hbhA | Rv0475 | Iron-regulated heparin- | 199 aa |
| | | | binding hemagglutinin | |
| Ex 10 | rpsD | Rv3458c | 30S ribosomal | 201 aa |
| | | | protein S4 | |
| Ex 11 | rplI | Rv0056 | 50S ribosomal | 152 aa |
| | | | protein L9 | |
| Ex 12 | rplJ | Rv0651 | 50S ribosomal | 178 aa |
| | | | protein L10 | |
| Ex 13 | rplL | Rv0652 | 50S ribosomal | 130 aa |
| | | | protein L7/L12 | |
| Ex 14 | rpmC | Rv0709 | 50S ribosomal | 77 aa |
| | | | protein L29 | |
| Ex 15 | Putative DNA | Rv2731 | Putative DNA | 450aa |
| | repair ATPase | | repair ATPase | |
| Ex 16 | hypothetical | Rv3169 | Hypothetical | 374aa |
| | protein | | protein MT3258 | |

References cited herein are listed below for convenience and are hereby incorporated by reference in their entirety.
1. Agamemnon, et al., The RNA degradosome of Escherichia coli: an mRNA-degrading machine assembled on RNase E., Annual Review of Microbiology (2007), Volume: 61, Pages: 71-87.
2. Andries, K., et al., A diarylquinoline drug active on the ATP synthase of Mycobacterium tuberculosis. Science, 2005. 307(5707): p. 223-7.
3. Barends, A. W. Karzai, R. T. Sauer, J. Wower, B. Kraal, J. Mol. Biol. 314, 9 (Nov. 16, 2001).
4. Boni, V. S. Artamonova, M. Dreyfus, J. Bacteriol. 182, 5872 (October, 2000).
5. Boshoff, H. I., et al., Biosynthesis and recycling of nicotinamide cofactors in mycobacterium tuberculosis. An essential role for NAD in nonreplicating bacilli. J Biol Chem, 2008. 283(28): p. 19329-41.
6. Boshoff, H. I., V. Mizrahi, and C. E. Barry, 3rd, Effects of pyrazinamide on fatty acid synthesis by whole mycobacterial cells and purified fatty acid synthase I. J Bacteriol, 2002. 184(8): p. 2167-72.
7. Bycroft, T. J. Hubbard, M. Proctor, S. M. Freund, A. G. Murzin, Cell 88, 235 (Jan. 24, 1997).
8. Byrne, et al., Aspirin and ibuprofen enhance pyrazinamide treatment of murine tuberculosis, J. Antimicrob. Chemother. (2007) 59 (2): 313-316.
9. Cheng, S. J., et al., pncA mutations as a major mechanism of pyrazinamide resistance in Mycobacterium tuberculosis: spread of a monoresistant strain in Quebec, Canada. Antimicrob Agents Chemother, 2000. 44(3): p. 528-32.
10. Diacon, A. H., et al., The diarylquinoline TMC207 for multidrug-resistant tuberculosis. N Engl J Med, 2009. 360(23): p. 2397-405.
11. Ibrahim, M., et al., Synergistic activity of R207910 combined with pyrazinamide against murine tuberculosis. Antimicrob Agents Chemother, 2007. 51(3): p. 1011-5.
12. Junop et al., EMBO J. 19, 5962 (Nov. 15, 2000).
13. Keiler, Annu Rev. Microbiol. 62, 133 (2008).
14. Lambert, G. Boileau, J. G. Howe, R. R. Traut, J. Bacteriol. 154, 1323 (June, 1983).
15. Li, R. Hirano, H. Tagami, H. Aiba, RNA 12, 248 (February, 2006).
16. McCune, R. and R. Tompsett, The fate of Mycobacterium tuberculosis in mouse tissues as determined by the microbial enumeration technique. I. The persistence of drug susceptible tubercle bacilli in the tissues despite prolonged antimicrobial therapy. J Exp Med, 1956. 104: p. 737-762.
17. McCune, R. M., et al., Microbial persistence. I. The capacity of tubercle bacilli to survive sterilization in mouse tissues. J Exp Med, 1966. 123(3): p. 445-68.
18. McCune, R. M., F. M. Feldmann, and W. McDermott, Microbial persistence. II. Characteristics of the sterile state of tubercle bacilli. J Exp Med, 1966. 123(3): p. 469-86.
19. McDermott, W. and R. Tompsett, Activation of pyrazinamide and nicotinamide in acidic environment in vitro. Am Rev Tuberc, 1954. 70: p. 748-754.
20. Mitchison, The action of antituberculosis drugs in short course chemotherapy. Tubercle, 1985. 66: p. 219-225.
21. Mitchison, et al., Recent Developments in the Study of Pyrzainamide: An Update, Prog Respir Res. Base1, Karger, 2011, vol 40, pp 1-12.
22. Muto et al., Genes Cells 5, 627 (August, 2000).
23. Nuermberger et al., Antimicrob. Agents Chemother. 52, 1522 (April, 2008).
24. Nuermberger, M. K. Spigelman, W. W. Yew, Respirology 15, 764 (July, 2010).
25. Oganesyan, N., S. H. Kim, and R. Kim, On-column protein refolding for crystallization. J Struct Funct Genomics, 2005. 6(2-3): p. 177-82.
26. Pethe, et al., The heparin-binding haemagglutinin of M. tuberculosis is required for extrapulmonary dissemination, Nature 412, 190-194 (12 Jul. 2001).
27. Rosenthal, I. M., et al., Daily dosing of rifapentine cures tuberculosis in three months or less in the murine model. PLoS Med, 2007. 4(12): p. e344.
28. Saguy, R. Gillet, P. Skorski, S. Hermann-Le Denmat, B. Felden, Nucleic Acids Res 35, 2368 (2007).
29. Scorpio, A. and Y. Zhang, Mutations in pncA, a gene encoding pyrazinamidase/nicotinamidase, cause resistance to the antituberculous drug pyrazinamide in tubercle bacillus. Nat Med, 1996. 2(6): p. 662-7.
30. Scorpio, A., et al., Characterization of pncA mutations in pyrazinamide-resistant Mycobacterium tuberculosis. Antimicrob Agents Chemother, 1997. 41(3): p. 540-3.
31. Somoskovi, A., et al., Iron enhances the antituberculous activity of pyrazinamide. J Antimicrob Chemother, 2004. 53(2): p. 192-6.
32. Suryanarayana, A. R. Subramanian, Biochemistry (Mosc). 22, 2715 (May 24, 1983).
33. Tarshis, M. S, and W. A. Weed, Jr., Lack of significant in vitro sensitivity of Mycobacterium tuberculosis to pyrazinamide on three different solid media. Am Rev Tuberc, 1953. 67(3): p. 391-5.
34. Tasneen, S. Tyagi, K. Williams, J. Grosset, E. Nuermberger, Antimicrob. Agents Chemother. 52, 3664 (October, 2008).
35. Thibonnier, J. M. Thiberge, H. De Reuse, PLoS One 3, e3810 (2008).
36. Valle et al., Science 300, 127 (Apr. 4, 2003).
37. Wade, M. M. and Y. Zhang, Anaerobic incubation conditions enhance pyrazinamide activity against Mycobacterium tuberculosis. J Med Microbiol, 2004. 53(Pt 8): p. 769-73.
38. Wade, M. M. and Y. Zhang, Effects of weak acids, UV and proton motive force inhibitors on pyrazinamide activity against Mycobacterium tuberculosis in vitro. J Antimicrob Chemother, 2006. 58(5): p. 936-41.
39. Wower, C. W. Zwieb, S. A. Guven, J. Wower, EMBO J. 19, 6612 (Dec. 1, 2000).
40. Yew, M. Cynamon, Y. Zhang, Expert Opin. Emerg. Drugs, (Sep. 26, 2010).
41. Zhang, W. W. Yew, Int. J. Tuberc. Lung Dis. 13, 1320 (November, 2009).
42. Zhang, Y. and D. Mitchison, The curious characteristics of pyrazinamide: a review. Int J Tuberc Lung Dis, 2003. 7(1): p. 6-21.
43. Zhang, Y., et al., Genetic analysis of superoxide dismutase, the 23 kilodalton antigen of Mycobacterium tuberculosis. Mol Microbiol, 1991. 5(2): p. 381-91.
44. Zhang, Y., et al., Mode of action of pyrazinamide: disruption of Mycobacterium tuberculosis membrane transport and energetics by pyrazinoic acid. J Antimicrob Chemother, 2003. 52(5): p. 790-5.
45. Zhang, Y., et al., Role of acid pH and deficient efflux of pyrazinoic acid in unique susceptibility of Mycobacterium tuberculosis to pyrazinamide. J Bacteriol, 1999. 181(7): p. 2044-2049.
46. Zhang, Y., et al., The catalase-peroxidase gene and isoniazid resistance of Mycobacterium tuberculosis. Nature, 1992. 358(6387): p. 591-3.
47. Zhang, Y., S. Permar, and Z. Sun, Conditions that may affect the results of susceptibility testing of Mycobacterium tuberculosis to pyrazinamide. J Med Microbiol, 2002. 51(1): p. 42-49.
48. Zimhony, O., et al., Pyrazinamide inhibits the eukaryotic-like fatty acid synthetase I (FASI) of Mycobacterium tuberculosis. Nat Med, 2000. 6(9): p. 1043-7.

1. A method of screening for an antibacterial agent that decreases persister formation or survival, eliminates or reduces bacterial infection or disease and/or increases killing of a bacterial cell, comprising the steps of:
(a) contacting a test agent with a composition comprising a Pyrazinamide (PZA)-sensitive target protein; and
b) determining whether the test agent binds to, or inhibits activity of, the target protein,
wherein binding of the target protein or inhibition of the target protein activity is indicative of a potential antibacterial agent for decreasing persister formation or survival, eliminating or reducing bacterial infection or disease and/or increasing killing of a bacterial cell.

2. The method of claim 1, wherein the agent binds to, and inhibits the activity of, the target protein.

3. The method of claim 1, wherein the target protein is selected from one or more of Endonuclease IV (Rv0670) Nfo (end) involved in DNA repair; Polynucleotide phosphorylase (PNPase) (Rv2783c) GpsI; Iron-regulated heparin-binding hemagglutinin (Rv0475) HbhA involved in extra-pulmonary dissemination of M. tuberculosis; 30S ribosomal protein S1 (Rv1630) RpsA; 30S ribosomal protein S4 (Rv3458c) RpsD; 50S ribosomal protein L9 (Rv0056) RplI; 50S ribosomal protein L10 (Rv0651) RpIJ; 50S ribosomal protein L7/L12(Rv0652) RpIL; 50S ribosomal protein L29 (Rv0709) RpmC; Putative DNA repair ATPase (Rv2731) and Hypothetical protein MT3258 (Rv3169).

4. The method of claim 1, wherein the target protein is a protein inhibited by Pyrazinamide (PZA) or pyrazinoic acid (POA).

5. The method of claim 1, wherein the target protein is a protein that binds, to or is inhibited by active component of PZA, Pyrazinoic Acid (POA), or an analog thereof.

6. The method of claim 1, wherein the POA-analog is 5-hydroxyl-2-pyrazinecarboxylic acid.

7. The method of claim 1, wherein the target protein is Endonuclease IV (Rv0670) nfo (end) and the inhibitory activity is Endonuclease V activity.

8. The method of claim 1, wherein the target protein is Endonuclease IV and the inhibitory activity is DNA repair.

9. The method of claim 1, wherein the target protein is HbhA and the inhibitory activity is iron-regulated heparin-binding involved in dissemination of M. tuberculosis in vivo.

10. The method of claim 1, 2 or 3, wherein the target protein is GpsI (PNPase) and the inhibitory activity is polynucleotide phosphorylation and ppGpp production involved in survival under starvation or stress conditions.

11. The method of claim 1, wherein the target protein is ATPase (Rv2731) and the inhibitory activity is DNA repair.

12. The method of claim 1, wherein the target protein is 30S ribosomal protein S1 (Rv1630) RpsA.

13. The method of claim 12, wherein the agent binds to the 30S ribosomal protein S1 (Rv1630) RpsA on the C-terminus.

14. The method of claim 12, wherein the agent binds to the 30S ribosomal protein S1 (Rv1630) RpsA at alanine residue 438 (AA438) in the C-terminus.

15. The method of claim 1, wherein the agent inhibits translation process, trans-translation process, or both.

16. The method of claim 1, wherein the agent binds to 30S ribosomal protein S1 (Rv1630) RpsA involved in trans-translation process.

17. The method of claim 1, wherein the agent inhibits EF-Tu function involved in trans-translation process.

18. The method of claim 1, wherein the agent inhibits SmpB function involved in trans-translation process.

19. The method of claim 1, wherein the agent inhibits tmRNA (SsrA) function involved in trans-translation process.

20. The method of claim 1, wherein the agent binds to 30S ribosomal protein S1 (Rv1630) RpsA and inhibits EF-Tu, tmRNA (SsrA) or SmpB function involved in trans-translation process.

21. The method of claim 1, wherein the target protein is obtained from a mutated version of a bacterial cell that is pyrazinamide (PZA) or Pyrazinoic Acid (POA) resistant.

22. The method of claim 21, wherein the mutated version of the bacterial cell is DHM444.

23. The method of claim 1, further comprising identifying PZA resistance in a bacterial cell, comprising the steps of:
(a) determining binding or inhibitory activity of PZA or POA in a wild type strain of said bacterial cell, and
(b) determining binding or inhibitory activity of PZA on a mutated strain of said bacterial cell,
wherein binding or inhibitory activity in the wild type version of the target proteins and lack of binding and inhibitory activity in the mutated version indicates PZA resistance.

24. The method of claim 23, wherein the wild type version is M. tuberculosis, and the mutated strain is DHM444.

25. The method of claim 1, further comprising screening for a second antibacterial agent for enhancing or synergizing the activity of the potential antibacterial agent, pyrazinamide (PZA) or Pyrazinoic Acid (POA), comprising the steps of
(a) contacting a second test agent that inhibits a second target protein selected from at least one of a protein encoded by nuoH, NADH dehydrogenase, protein encoded by nuoN, NADH dehydrogenase, protein encoded by fdhF, formate dehydrogenase α-subunit, protein encoded by narH, nitrate reductase β-subunit, protein encoded by pncB1, nicotinatephosphoribosyltransferase, protein encoded by yjcE, Na/H exchanger, or protein encoded by kdpA, potassium transporting ATPase; and
(b) determining whether the second test agent binds to, or inhibits activity of, the second target protein,
wherein binding of the second target protein or inhibition of the second target protein activity is indicative of a second potential antibacterial agent for enhancing or synergizing the activity of the potential antibacterial agent, pyrazinamide (PZA) or Pyrazinoic Acid (POA).

26. The method of claim 1, wherein the target protein is coupled to a solid support coated with target proteins for screening the test agent.

27. The method of claim 26, wherein the solid support is a gel column or beads.

28. The method of claim 26, comprising a technique selected from one or more of an affinity chromatographic matrix, target enzyme assays, or trans-translation assay that uses antibody to react with the tags produced from trans-translation.

29. The method of claim 26, further comprising a control column solid support that is coupled with ethanolamine without the target protein.

30. The method of claim 26, wherein a test solution is prepared containing the test agent.

31. The method of claim 30, wherein the test solution is added and the sample is run and washed with buffer to minimize nonspecific binding of proteins.

32. The method of claim 31, wherein a sample containing test agent bound to target protein is eluted by ethylene glycol and the fractions are run on SDS-PAGE gels and stained.

33. The method of claim 32, wherein the test agent is excised and subjected to in-gel digestion with trypsin followed by analysis by Ion Trap Tandem Mass Spectrometry to determine the identity of the test agent.

34. The method of claim 1, wherein persister or bacterial infection comprises one or more of latent infections, chronic and recurrent infections, and biofilm infections.

35. The method of claim 1, wherein the disease is selected from one or more of Tuberculosis, Lyme disease, Syphilis, Peptic ulcer, Bacteremia/Sepsis, Endocarditis, Otitis media, Urinary tract infections, Brucellosis, and Biofilm infections.

36. The method of claim 1, wherein the bacteria or pathogen is selected from one or more of M. tuberculosis, Borrelia burgdorferi, Treponema pallidum, H. pylori, S. aureus, Group A and Group B Streptococcus, Streptococcus species, Staphylococcus, enterococcus, S. pneumoniae, H. Influenzae, Moraxella catarrhalis, Klebsiella, E. coli, Chlamydia, Mycoplasma, M. genitalium, and Brucella abortus.
